(19) United States
(12) Patent Application Publication
(10) Pub. No.: US 2026/0272698 A1
(43) Pub. Date: Sep. 17, 2026

(54) METHOD FOR HAPTIC FEEDBACK FOR OSTOMY LEAKAGE DETECTION SYSTEM

(71) Applicant: Hollister Incorporated, Libertyville, IL (US)

(72) Inventors: Jonas P. Carlsson, Chicago, IL (US); Kyle A. Matthews, Chapel Hill, NC (US); Anthony B. Smith, Durham, NC (US); Scott E. Liddle, Raleigh, NC (US)

(73) Assignee: Hollister Incorporated, Libertyville, IL (US)

(21) Appl. No.: 19/469,020

(22) PCT Filed: May 15, 2024
(86) PCT No.: PCT/US2024/029398

§ 371 Date: Sep. 25, 2025

Related U.S. Application Data
(60) Provisional application No. 63/510,679, filed on Jun. 28, 2023.

(51) Int. Cl. A61F 5/44; A61F 5/445

(52) U.S. Cl. CPC A61F 5/4404; A61F 5/445

(57) ABSTRACT

A method for haptic feedback for an ostomy leakage detection system can include a wearable device to obtain leakage data. The leakage data may include leakage detected on at least one ring of a sensing accessory. The wearable device can also determine a leakage level based on the leakage data. The leakage level can indicate information about the leakage detected. The wearable device can further output a haptic feedback leak level alert based on the leakage level. The haptic feedback leak level alert can discreetly notify a user about the leakage detected.

### BACKGROUND

This disclosure is related to an ostomy leakage detection system. More particularly, the present disclosure pertains to a method for haptic feedback for an ostomy leakage detection system.

Known ostomy pouch systems can include a pouch formed from opposing sidewalls defining an internal collection area, an inlet opening for receiving a stoma, and an ostomy appliance for attaching the pouch to a user. Commonly, such ostomy appliance may include, for example, an ostomy barrier of a one-piece pouch system which can be attached to one of the pouch sidewalls proximate an inlet opening, or a faceplate for a two-piece pouch system configured to releasably engage a pouch, and a barrier ring. The ostomy appliance may include a skin barrier material for adhering to and sealing against a user's peristomal skin surrounding the stoma.

However, the ostomy appliance may be susceptible to ostomy effluent leakage, and the seal formed between the skin barrier material and the user may weaken. Oftentimes, the user may be unaware of, or cannot easily assess, the extent of weakening in the seal. Thus, the user may not become aware of a weakened seal, and consequently, the ostomy effluent may leak through to an exterior of the ostomy appliance. An ostomy leakage detection system may be used to detect such a leak. Such ostomy leakage detection systems can include subsystems that comprise a wearable device for alerting a user of an ostomy pouch system to the presence of ostomy effluent (or leakage) under a hydrocolloid skin barrier. However, alerting the user of a leakage detection or information about the wearable device may be limited to sound or light notifications that may not be sufficiently discreet or adequate to notify a user. For example, the wearable device may discreetly alert user of a leakage detection by displaying a light, but that light may not be visible under the user's clothing or it may be visible to others near the user.

Accordingly, it is desirable to provide an improved ostomy detection system that provides discrete alerts to the user.

### BRIEF SUMMARY

A method for haptic feedback for an ostomy leakage detection system is provided according to various embodiments.

In a first aspect of the present disclosure, a method for haptic feedback for an ostomy leakage detection system is provided.

In an embodiment, the method may be applied to a wearable device. The wearable device may obtain leakage data. The leakage data may include a leakage detected on at least one ring of a sensing accessory. The wearable device may also determine a leakage level based on the leakage data. The leakage level indicates information about the leakage detected. The wearable device may further output a haptic feedback leak level alert based on the leakage level. The haptic feedback leak level alert discreetly notifies a user about the leakage detected.

In an embodiment, the wearable device may determine the leakage level based on the leakage data and a predefined ring threshold. In such an embodiment, the haptic feedback leak level alert may include at least one haptic pulse based on the leakage level. The haptic feedback leak level alert may include a single haptic pulse when the leakage level equals to one. The haptic feedback leak level alert may include three haptic pulses when the leakage level equals to three. The haptic feedback leak level alert may include a different haptic strength based on the leak level. The haptic feedback leak level alert may include a soft haptic pulse when the leakage level equals to one. The haptic feedback leak level alert may include a stronger haptic pulse when the leakage level equals to three.

In an embodiment, the wearable device may determine a double tap was detected. The wearable device may also output a haptic feedback device status alert based on the double tap. In such an embodiment, the haptic feedback device status alert may include a ramping pattern.

In an embodiment, the wearable device may determine a battery level. The wearable device may also output a haptic feedback battery level alert. In such an embodiment, the battery level may include a critical low battery level. The haptic feedback battery level alert may include a short and a long pulse outputted twice.

In an embodiment, the wearable device may determine a device has been lost. The wearable device may also output a haptic feedback lost device alert. In such an embodiment, the haptic feedback lost device alert may include six short pulses.

In a second aspect of the present disclosure, a computing device is provided. The computing device may include one or more processors, a non-transitory computer-readable memory storing instructions executable by the one or more processors. The one or more processors may be configured to obtain a leakage data. The leakage data may include a leakage detected on at least one ring of a sensing accessory. The one or more processors may also be configured to determine a leakage level based on the leakage data. The leakage level indicates information about the leakage detected. The one or more processors may further be configured to output a haptic feedback leak level alert based on the leakage level. The haptic feedback leak level alert discreetly notifies a user about the leakage detected.

In an embodiment, one or more processors may also be configured to determine the leakage level based on the leakage data and a predefined ring threshold. In such an embodiment, the haptic feedback leak level alert may include three haptic pulses when the leakage level equals to three. The haptic feedback leak level alert may include a different haptic strength based on the leak level.

In a third aspect of the present disclosure, a non-transitory computer-readable storage medium having instructions stored therein is provided. When the instructions are executed by one or more processors of the apparatus, the instructions may cause the apparatus to obtain leakage data. The leakage data may include a leakage detected on at least one ring of a sensing accessory. The instructions may also cause the apparatus to determine a leakage level based on the leakage data and a predefined ring threshold. The leakage level indicates information about the leakage detected. The instructions may further cause the apparatus to output a haptic feedback leak level alert based on the leakage level. The haptic feedback leak level alert discreetly notifies a user about the leakage detected.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1A is an illustration of an ostomy leakage detection system according to an embodiment.

FIG. 1B is a front view of an ostomy system attached to a user, according to an embodiment.

FIG. 2A is a body-side perspective view of a sensor circuit, according to an embodiment.

FIG. 2B is a partially enlarged body-side perspective view of a sensor circuit, according to an embodiment.

FIG. 3 is a top-side perspective view of a wearable device in a closed position according to an embodiment.

FIG. 4 is a top partial cross-sectional perspective view of the wearable device of FIG. 2 in an open position.

FIG. 5 is a flow diagram illustrating a method for haptic feedback for an ostomy leakage detection system, according to an embodiment.

FIG. 6 is a flow diagram illustrating a method for haptic feedback for an ostomy leakage detection system, according to another embodiment.

FIG. 7 is a schematic illustration of a computing environment, according to an embodiment.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,“ ”down,“ front,” “back,” and the like are used for purposes of illustration and as such, are not limiting. Depending on the context, the word “if” as used herein may be interpreted as “when” or “upon” or “in response to determining.” The present disclosure provides methods for haptic feedback for ostomy leakage detection system. Haptic feedback can be a process by which a motor causes a vibration of a device that can be physically felt by the user. Normally, haptics are used as a ‘silent’ notification to notify the user of an event without alerting others. The ostomy leakage detection systems can be configured to detect ostomy effluent leakage under a skin barrier and to alert a user. The ostomy leakage detection systems can provide multiple benefits to the user. For example, the systems can allow the user to intervene and change a skin barrier and/or ostomy pouch system before a leak progresses, which can cause embarrassment and inconvenience to the user. Further, the ostomy leakage detection systems can assist in maintaining a user's skin health by detecting and communicating a leakage in its early stage to a user to prevent prolonged skin exposure to ostomy effluent, which can lead to skin health complications. The ostomy leakage detection systems can also support a user's emotional well-being by reducing anxiety associated with a risk of leakage. The ostomy leakage detection systems may be applied to an ostomy barrier of a one-piece pouch system or a faceplate of a two-piece pouch system.

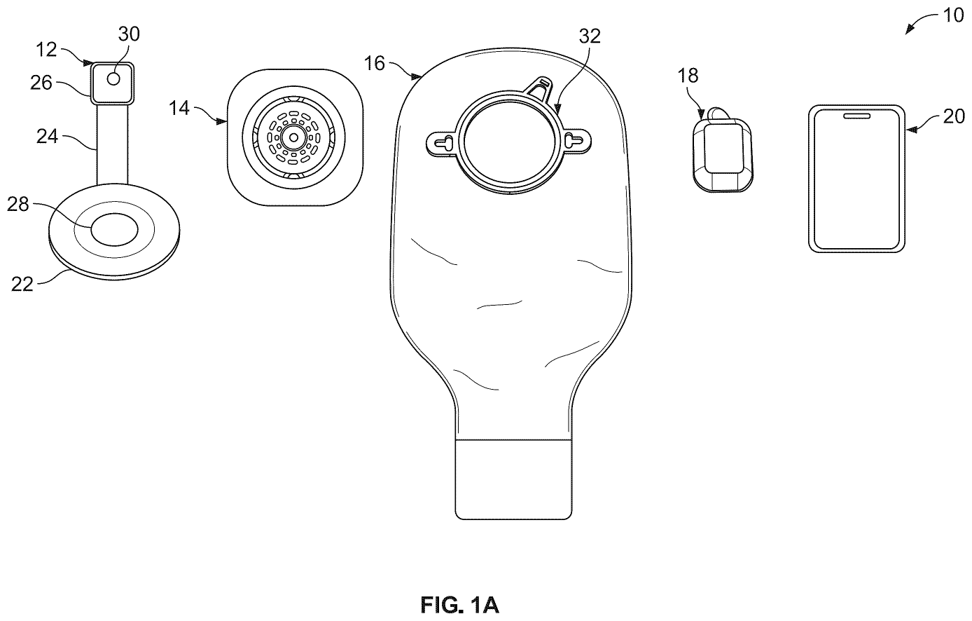

FIG. 1 illustrates an ostomy two-piece pouch system 10. According to example embodiments shown schematically in FIG. 1, the ostomy system 10 can generally include a sensing accessory 12, an ostomy barrier appliance 14, an ostomy bag 16, a wearable device 18, and a mobile device 20. The sensing accessory 12 can include a sensing region 22, a tail region 24, and a connection region 26. The sensing region 22 can include an inlet opening 28 configured to surround a stoma (not shown). The tail region 24 can include a connector opening 30 configured to electrically and mechanically connect with the wearable device 18. The ostomy bag 16 can receive and hold bodily waste and may include an ostomy barrier coupling member 32 configured to attach to the sensing region 22.

FIG. 1B illustrates the ostomy pouch system 10 attached to a user according to example embodiments. According to example embodiments shown schematically in FIG. 1B, the sensing accessory 12 can be mounted to a user using an adhesive and the inlet opening 28 can surround the stoma. The ostomy barrier appliance 14 can be mounted over the sensing region 22 with an adhesive and can surround the stoma. The ostomy pouch 16 can be mounted on the ostomy barrier appliance 14 using the pouch coupling member 32. The wearable device 18 can be attached to the sensing accessory 12 using the connector opening 30. The wearable device 18 can be mounted on a user using a patch or an adhesive.

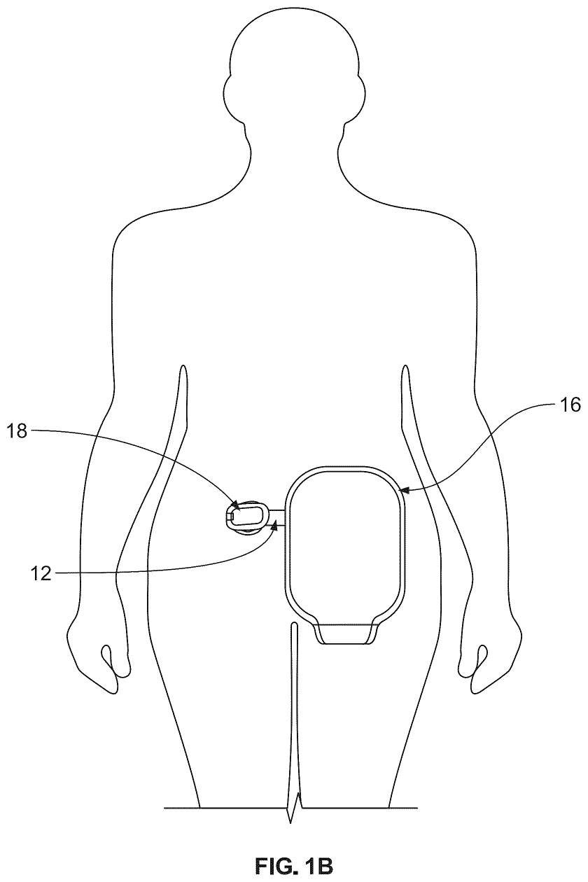

According to example embodiments, the ostomy leakage detection system may comprise three subsystems-the sensing accessory 12, the wearable device 20, and a mobile application on the mobile electronic device 21. The sensing accessory 12 may be provided as an accessory for an ostomy pouch system. The sensing accessory 12 can include sensors for detecting the presence of ostomy effluent. The sensing accessory 12 may be configured to communicate leakage detection signals to the wearable device 20. For example, the wearable device 20 can generate leakage data based on the leakage detection signals from the sensing accessory 12. Referring to FIG. 2A, the leakage data can include whether leakage is detected and in what ring and quadrant the leakage is detected.

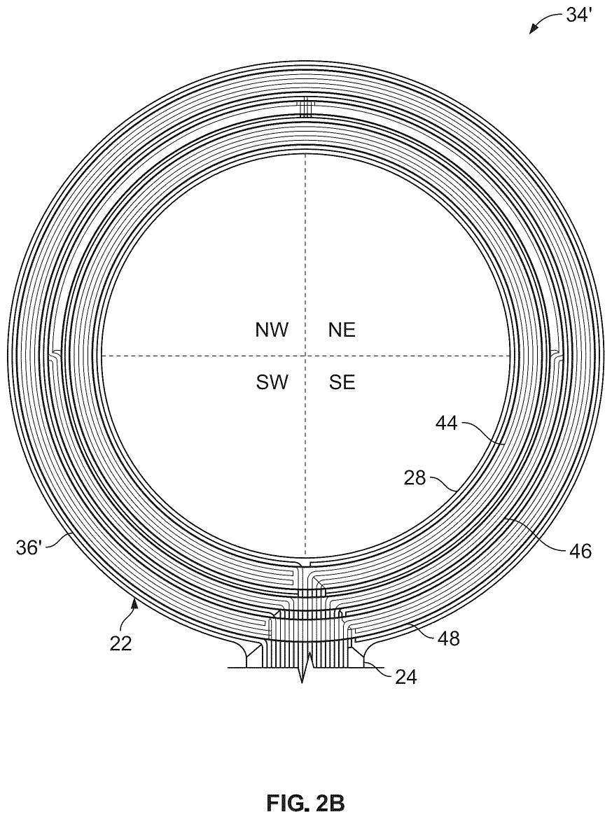

FIG. 2A shows a body-side view of a sensor circuit 34. According to example embodiments shown schematically in FIG. 2A, the sensor circuit 34 can generally include a conductive sensor 36, a conductive trace 38, and a connection point 40. The sensor circuit 34 can be located on the sensing region 22, the tail region 24, and the connection region 26. The conductive sensor 36 can be configured to detect a leak around a stoma. The conductive trace 38 can be located on the tail region 24 and may be configured to connect the conductive sensor 36 to the connection point 40. The connection point 40 can be located on the connection region 26 and may be configured to connect to a connection pad 31 (FIG. 4) on the wearable device 20. In one or more embodiments, the sensing region 22 is broken into four quadrants NE, SE, NW, SW.

According to example embodiments, the conductive sensor 36 can include multiple pairs of conductive traces that can be arranged in radial sensing levels (or rings) with levels broken into the four quadrants NE, SE, NW, SW of sensor pairs for detecting the direction of fluid and moisture progression. The radial sensing levels can include a first radial sensing level (or first ring 44) located near the inlet opening 28, a second radial sensing level (or second ring 48) located near the outer edge of the sensing region 22, and one or more radial sensing levels (or rings 46) located between the first and second radial sensing levels. The conductive traces can be adjacent to ground traces so that resistance may be measured between the two for detecting fluid and moisture. In another embodiment, the conductive traces can detect fluid and moisture based on resistance, resonance frequency or the like.

The conductive sensor 36 may be arranged in a predetermined pattern in the sensing region 22. For example, the sensor circuit 34 may be generally arranged in a circular or semi-circular pattern. The sensor circuit 34 in the sensing region 22 may be arranged at one or more radial distances from the inlet opening 28. For example, the sensor circuit 34 may include a plurality of electrically conductive traces arranged at a plurality of different radial distances from the inlet opening 28. The sensor circuit 34 may include conductive traces and conductive pads or points that may be formed by printing on a circuit substrate using a conductive ink via a conventional printing process, for example, screen printing. In another embodiment, the conductive sensor 36 can include other suitable patterns, such as an oval or oblong pattern, or other closed or substantially closed loop patterns.

FIG. 2B shows a body-side view of a sensor circuit 34′. According to example embodiments shown schematically in FIG. 2B, the sensor circuit 34′ can include a conductive sensor 36′. The conductive sensor 36′ can include a first radial sensing level 44 (or first ring) located near the inlet opening 28, a second radial sensing level 46 (or second ring) located between the first radial sensing level and a third radial sensing level, and the third radial sensing levels 48 (or third ring) located near the outer edge of the sensing region 22.

### Leakage Detection

In an embodiment, the wearable device 20 can detect leakage. For example, the leakage data can be generated by the wearable device 20 using electrical signals from the sensing accessory 12. The leakage data can include whether a leakage is detected and in what ring and quadrant the leakage is detected. The leakage level can then be determined based on predefined ring thresholds. The predefined ring thresholds can help indicate to the user how a leak is progressing from the inlet opening 28. The leakage level can be determined on the wearable device 20, the mobile application on the mobile electronic device 21, or other computing system that has access to the leakage data.

In an embodiment, the predefined ring thresholds can include a threshold for each ring detecting a leakage. For example, a first ring threshold can be met when a first ring detects a leakage. The leakage level can then be determined to be a level 1. In another example, a second ring threshold can be met when two rings detect a leakage, and the leakage level can be determined to be a level 2. In yet another example, a third ring threshold can be met when all rings detect a leakage, and the leakage level can be determined to be a level 3.

In another embodiment, the predefined ring thresholds can be set not only based on the number of rings detecting a leakage, but which rings detect such a leakage. For example, a leakage level 1 can include a predefined ring threshold for an inner ring that indicates that a leak is detected only on the inner ring of the sensing region 22, a leakage level 2 can include a predefined ring threshold for an inner ring and middle ring that indicates that the leak is detected on the inner ring and is progressing onto the middle ring, and the leakage level 3 can include a predefined ring threshold for an inner ring, middle ring, and outer ring that indicates that the leak is detected on the inner ring and has progressed onto the middle ring and the outer ring.

In another embodiment, a moisture level can be determined. The leakage data can include whether moisture is detected and in what ring and quadrant the moisture is detected. A moisture level can then be determined based on predefined ring thresholds. For example, the moisture level can include a predefined ring threshold for an outer ring that indicates that moisture is detected only on the outer ring of the sensing region 22.

### Wearable Device

The wearable device 20 may be configured to perform at least some processing of the leakage detection signals and alert a user of a leakage event. The wearable device 20 may be configured to electronically communicate with the mobile application through a wired or wireless communication system. Such electronic communications may include raw data acquired from the sensing accessory 12 or summarized leak data generated from processing the raw data. The wearable device 20 may also communicate system conditions, such as the presence of a sensing accessory, a faulty sensor, or a battery state. The mobile application may be a digital subsystem and/or software application installed and able run on the mobile electronic device 21. The mobile application may be configured to further process leak detection data and provide an alert or other information about an ostomy appliance to a user.

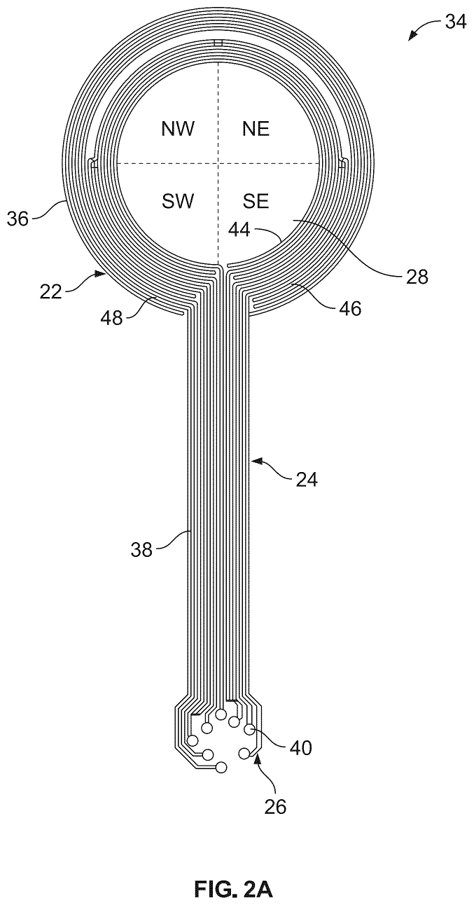

Referring to figures, FIGS. 3 and 4, a wearable device 20 according to an embodiment is illustrated. The wearable device 20 may generally include a top housing 50, a bottom housing 52, a hinge 54, a lip 56, a latching mechanism 58, and one or more light indicators 60.

FIG. 3 shows the wearable device 20 in a closed position. The lip 56 can be located on an edge of the top housing 50 and opposite the hinge 54. According to example embodiments shown schematically in FIG. 3, the lip 56 can aid in opening and closing the wearable device 20. The lip 56 may be a touch point for opening the top housing 50. The latching mechanism 58 can secure the wearable device 20 in the closed position. The one or more light indicators 60 can be viewable from the top housing 50 and configured to indicate conditions of the wearable device 20 such as a system status, battery status, and error messages. In another embodiment, the one or more light indicators 60 can be positioned at the sides of the wearable device 20 or other areas where they can be visible to a user while wearing the wearable device 20. For example, one or more light indicators 60 can light the entire top housing 50 by having the top housing 50 being made of a translucent material as the cover and positioning the one or more light indicators 60 under the top housing 50 to light top housing 50.

FIG. 4 shows a top perspective partial cross-sectional view of the wearable device 20 in an open position. According to example embodiments shown in FIG. 4, the wearable device 20 can include a printed circuit board (PCB) 62, a battery 64, and a haptic motor 66 within a sealed electronic enclosure. The haptic motor 66 can provide a haptic buzzer for silent or discreet notifications where the user, while wearing the wearable device 20 will feel vibrations from the haptic motor 66 in the form of pulses. The battery 64 or haptic motor 66 may be aligned within a center raised member for space savings. The PCB 62 can include a processing unit, a communication unit, and electronic connectors that connect with the one or more sensor contact pads 40. The PCB 62 can analyze signals received from the sensing accessory 12, communicate with a mobile device 21 or a charging dock, and alert a user through sound and vibration, as well as one or more lights to indicate a system status.

The haptic motor 66 can provide a pulse of different strength and durations. The haptic strength can include a strong, medium, or low strength based on the speed of the haptic motor 66. For example, a strong haptic strength can be affected by a fast motor speed, a medium haptic strength can be affected by a medium motor speed, and a low haptic strength can be effected by a slow motor speed.

### Haptic Feedback

In one or more embodiments, the haptic motor 66 can provide haptic feedback alerts. The haptic motor 66 can provide physical vibration feedback during use for leakage level alerting, device status, battery level alerts, and a lost device alert. The haptic motor 66 can be used by the wearable device 20 to notify the user that their attention is required. Wearable devices can use a combination of LEDs, speakers, or screens that can make up the primary communication mechanism to the user. However, the wearable device 20 can also deliver discreet notifications without alerting non-users nearby. This can remove the limitation of the wearable device 20 to utilize any notification paradigms that require the user to view the device itself (for example, by looking at the one or more light indicators 60), and screens or touchscreens (for example, on mobile device 21).

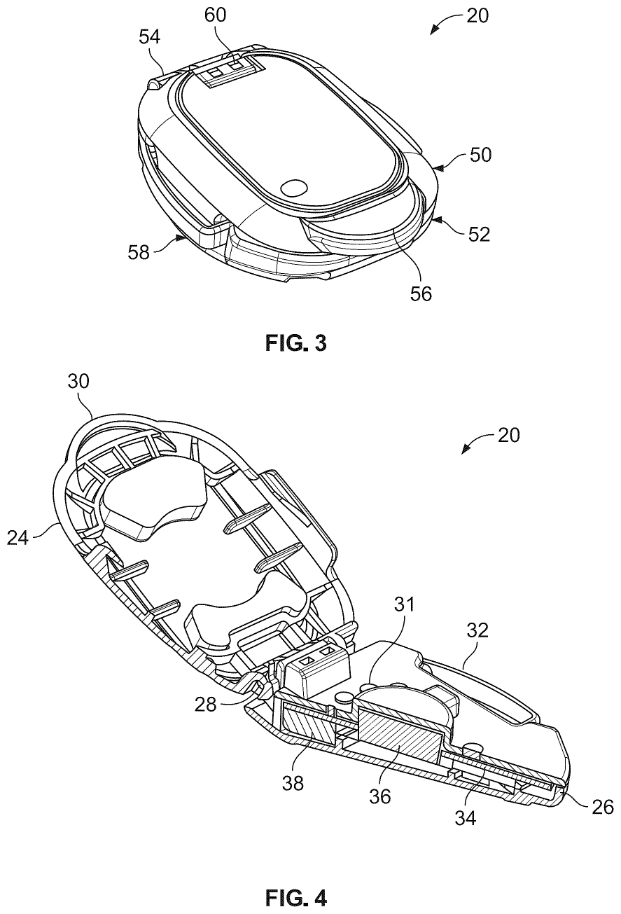

FIG. 5 shows method 500 for outputting a leakage level alert. The method may be applied to a computing device such as a wearable device, mobile device, personal computer or server.

In step 510, the wearable device can obtain leakage data. The leakage data can include whether leakage is detected and in what ring and quadrant the leakage is detected. The leakage data can further include whether moisture is detected and in what ring and quadrant the moisture is detected.

In step 520, the wearable device can determine a leakage level based on the leakage data. The leakage level can inform a user about the severity of the leakage detected. For example, the leakage level can be a leakage level 1 that may not be severe but should be noted by the user. In another example, the leakage level can be a leakage level 2, that may signal a progressing leakage that should be inspected by the user. In yet another example, the leakage level can be a leakage level 3, that may signal a severe leakage that requires the changing of the ostomy pouch system before a leak progresses and causes embarrassment and inconvenience to the user.

In an embodiment, the haptic strength and feedback during leak events can help illustrate the severity of the leak. For example, the strength of vibration can be changed (while keeping the pattern the same) based on an estimated certainty that a leakage is detected by the wearable device 20.

In step 530, the wearable device can output a haptic feedback leak level alert based on the leakage level. For example, the haptic feedback can include a haptic vibration at a set interval and pattern according to the leak severity (one haptic pulse per leak level, with a lull time in between pulse trains). That is the patterns of pulses varying by amplitude, duration, and/or repetition can be used so that each category of feedback can be differentiated by the user. For example, such an alert can be, leak level 1—single pulse, ~8 second lull, repeated until double tap or timeout, leak level 2—two pulses separated by 1 second, ~8 second lull, repeated until double tap or timeout, and leak level 3—three pulses separated by 1 second, ~8 second lull, repeated until double tap or timeout.

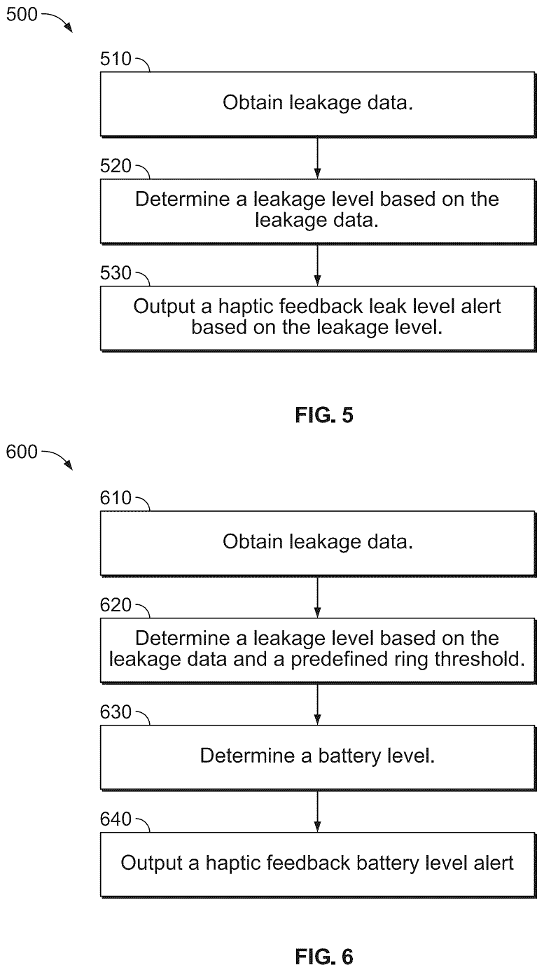

FIG. 6 shows a method 600 for outputting a leakage level alert. The method may be applied to a computing device such as a wearable device, mobile device, personal computer or server.

In step 610, the wearable device can obtain leakage data.

In step 620, the wearable device can determine a leakage level based on the leakage data and a predefined ring threshold.

In step 630, the wearable device can determine a battery level. The battery level can include a full battery level, normal battery level, battery low level, and a battery critical level. The full battery level can be a battery level above 90%. The normal battery level can be a battery level above 20% but below or equal to 90%. The battery low level can be a battery level above 5% but below or equal to 20%. The battery low level can signal to the user that the battery should be charged soon. The battery critical level can be a battery level below or equal to 5%. The ‘battery critical level can signal to the user that the battery should be charged immediately.

In step 640, the wearable device can output a haptic feedback battery level alert. The haptic feedback battery level alert can be output based on the battery level. For example, when the battery level is determined to be at a battery low level, the haptic feedback battery level alert is output. The haptic feedback battery level alert can include two short pulses separated by 1 second and repeated twice with an 8 second lull to indicate to the user low battery. In another example, when the battery level is determined to be at a battery critical level, the haptic feedback battery level alert is output. The haptic feedback battery level alert can include a short and a long pulse separated by 1 second and repeated twice with an 8 second lull to indicate to the user a low battery.

In another embodiment, the wearable device can output a haptic feedback device status alert based on a user input such as a double tap on the top housing 50 or input on the mobile device 21. The haptic feedback device status alert can include a ramping pattern haptic output. For example, the ramping pattern haptic output can include a short and long pulse seperated by 0.5 seconds and delivered once.

In another example, the system status of the wearable device 20 can determine the haptic feedback output. For example, if the system status of the wearable device 20 is in a training mode, the haptic feedback output can output a haptic vibration at a set interval to mimic a leak event of a specified severity. This training mode is entered upon receipt of a command from the mobile application on the mobile electronic device 21. If training mode is active and a double tap is detected, the training haptic pattern will shut off and the phone will be notified that the user double tapped. This mode is purely to train the user on how the haptic feels for a given severity of leak, and how to interact with the device via taps. This may be instructive, as many users are not used to tapping on a device, nor are they used to only receiving feedback via haptics from a device. Both of these use-flows (input and output) for the wearable device 20 help provide a discreet means of interaction.

In another embodiment, the wearable device can output a haptic feedback lost device alert. The haptic feedback lost device alert can include, for example, 6 haptic pulses in short succession (0.5 seconds), repeated after 8 second lulls until found by the user or timed out. A lost device can be determined based on the mobile electronic device 21 losing a network connection with the wearable device 20. In another example, the user can input to the application on the mobile electronic device 21 that the wearable device 20 has been lost.

In another embodiment, the wearable device can output a haptic feedback context-aware alert based on a context-aware event, such as when a user is swimming, walking or running. The context-aware event can be determined based on the wearable device detecting moisture that can indicate swimming or sweat and an accelerometer detecting movement consistent with swimming, walking or running. The haptic feedback context-aware alert can include a specific pulse pattern to notify the user that the device has detected swimming, walking or running and that it has adjusted itself accordingly. For example, the wearable device may increase its monitoring of leakage by increasing the sampling rate of data received from the sensing accessory 12 to detect leakage.

In another embodiment, the haptic patterns and strength can be set by a user for specific events using a mobile application on the mobile electronic device 21. For example, a user can shut off haptic patterns for specific events they consider less important or increase the strength of the haptic pulse when they are extra sensitive about leakage. The user can also change the haptic patterns and strength for modes such as sleep mode and daytime mode. For example, in sleep mode, haptic strength can be set as strong and continue until the user has awoken (via context-aware feedback from the accelerometer or by a current time) and entered daytime mode.

The above pulse patterns are examples meant to illustrate the significance of being able to customize the pattern according to the action that needs to be notified. Additionally, there is also importance to being able to change parameters other than the number of repetitions (i.e., haptic feedback where there is a single base pulse that is repeated a specific number of times for a specific event is only debatably helpful, as the users have a more difficult time parsing out the action).

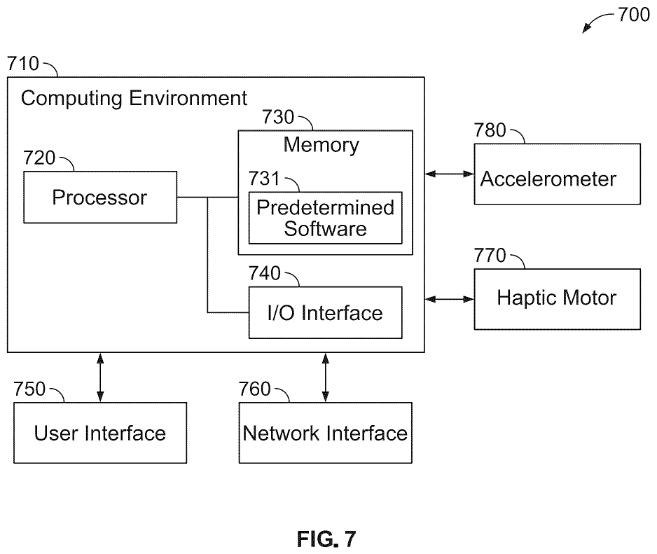

FIG. 7 shows a computing system 700 that can be part of the wearable device 20. According to example embodiments shown schematically in FIG. 7, the computing system 700 can include a computing environment 710, a user interface 750, a network interface 760, a haptic motor 770, and an accelerometer 780. The computing environment 710 can include a processor 720, a memory 730, and an I/O interface 740. The computing environment 710 can be coupled to the user interface 750, network interface 760, haptic motor 770, and accelerometer 780 through the I/O interface 740. The PCB 62 can include the computing environment 710.

The processor 720 can typically control the overall operations of the computing environment 710, such as the operations associated with data acquisition, data processing, and data communications. The processor 720 can include one or more processors to execute instructions to perform all or some of the steps in the above-described methods. Moreover, the processor 720 can include one or more modules that facilitate the interaction between the processor 720 and other components. The processor may be or include a Central Processing Unit (CPU), a microprocessor, a single chip machine, a graphical processing unit (GPU) or the like.

The memory 730 can store various types of data to support the operation of the computing environment 710. Memory 730 can include predetermined software 731. Examples of such data comprise instructions for any applications or methods operated on the computing environment 710, raw data, leak data, moisture data, resistance values, etc. The memory 730 may be implemented by using any type of volatile or non-volatile memory devices, or a combination thereof, such as a static random-access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic or optical disk.

The I/O interface 740 can provide an interface between the processor 720 and peripheral interface modules, such as a RF circuitry, external port, proximity sensor, audio and speaker circuitry, video and camera circuitry, microphone, accelerometer, display controller, optical sensor controller, intensity sensor controller, other input controllers, keyboard, a click wheel, buttons, and the like. The buttons may include but are not limited to, a home button, a power button, and volume buttons.

The user interface 750 can include a speaker, lights, display or other similar technologies for communicating with the user.

Network interface 760 provides communication between the processing unit, an external device, mobile device, and a webserver (or cloud). The communication can be done through, for example, WIFI or BLUETOOTH hardware and protocols. The network interface 760 can be within the computing environment or connected to it.

The haptic motor 770 can include a haptic feedback controller or driver that controls the haptic motor 66 and can cause a vibration or pulse on the wearable device 20. The vibration or pulse on the wearable device 20 can be physically felt by the user when worn or can generate a noise when the wearable device 20 is not worn but on a surface (for example, a table). The haptic feedback controller or driver can send electrical signals to control the haptic motor 770 and cause vibrating pulses.

The accelerometer 780 can include an electromechanical device or other similar technologies that measures a physical acceleration experienced by an object on one, two, or three axes. The accelerometer 780 can include, for example, capacitive plates attached to spring that move internally as acceleration forces act upon the sensor.

In some embodiments, there is also provided a non-transitory computer-readable storage medium comprising a plurality of programs, such as comprised in the memory 730, executable by the processor 720 in the computing environment 710, for performing the above-described methods. For example, the non-transitory computer-readable storage medium may be a ROM, a RAM, or the like.

The non-transitory computer-readable storage medium has stored therein a plurality of programs for execution by a computing device having one or more processors, where the plurality of programs when executed by the one or more processors, cause the computing device to perform the above-described method for motion prediction.

In some embodiments, the computing environment 710 may be implemented with one or more application-specific integrated circuits (ASICs), digital signal processors (DSPs), digital signal processing devices (DSPDs), programmable logic devices (PLDs), field-programmable gate arrays (FPGAs), graphical processing units (GPUs), controllers, micro-controllers, microprocessors, or other electronic components, for performing the above methods.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A method for haptic feedback for an ostomy leakage detection system comprising:
obtaining a leakage data, wherein the leakage data comprises a leakage detected on at least one ring of a sensing accessory;
determining a leakage level based on the leakage data, wherein the leakage level indicates information about the leakage detected; and
outputting a haptic feedback leak level alert based on the leakage level, wherein the haptic feedback leak level alert discreetly notifies a user about the leakage detected.

2. The method of claim 1, wherein determining the leakage level based on the leakage data comprises:
determining the leakage level based on the leakage data and a predefined ring threshold.

3. The method of claim 1, wherein the haptic feedback leak level alert comprises at least one haptic pulse based on the leakage level.

4. The method of claim 1, wherein the haptic feedback leak level alert comprises a single haptic pulse when the leakage level equals to one.

5. The method of claim 1, wherein the haptic feedback leak level alert comprises three haptic pulses when the leakage level equals to three.

6. The method of claim 1, wherein haptic feedback leak level alert comprises a different haptic strength based on the leak level.

7. The method of claim 1, wherein the haptic feedback leak level alert comprises a soft haptic pulse when the leakage level equals to one.

8. The method of claim 1, wherein the haptic feedback leak level alert comprises a strong haptic pulse when the leakage level equals to three.

9. The method of claim 1, further comprising:
determining a double tap was detected; and
outputting a haptic feedback device status alert based on the double tap.

10. The method of claim 9, wherein the haptic feedback device status alert comprises a ramping pattern.

11. The method of claim 1, further comprising:
determining a battery level; and
outputting a haptic feedback battery level alert.

12. The method of claim 11, wherein the battery level comprises a critical low battery level.

13. The method of claim 12, wherein the haptic feedback batter level alert comprises a short and long pulse outputted twice.

14. The method of claim 1, further comprising:
determining a device has been lost; and
outputting a haptic feedback lost device alert.

15. The method of claim 14, wherein the haptic feedback lost device alert comprises six short pulses.

16. A computing device, comprising:
one or more processors;
a non-transitory computer-readable storage medium storing instructions executable by the one or more processors, wherein the one or more processors are configured to:
obtain a leakage data, wherein the leakage data comprises a leakage detected on at least one ring of a sensing accessory;
determine a leakage level based on the leakage data, wherein the leakage level indicates information about the leakage detected; and
output a haptic feedback leak level alert based on the leakage level, wherein the haptic feedback leak level alert discreetly notifies a user about the leakage detected.

17. The computing device of claim 16, wherein the one or more processors configured to determine the leakage level based on the leakage data are further configured to:
determine the leakage level based on the leakage data and a predefined ring threshold.

18. The computing device of claim 1, 17, wherein the haptic feedback leak level alert comprises three haptic pulses when the leakage level equals to three.

19. The computing device of claim 1, 18, wherein the haptic feedback leak level alert comprises a different haptic strength based on the leak level.

20. A non-transitory computer-readable storage medium storing a plurality of programs for execution by a computing device having one or more processors, wherein the plurality of programs, when executed by the one or more processors, cause the computing device to perform acts comprising:
obtaining a leakage data, wherein the leakage data comprises a leakage detected on at least one ring of a sensing accessory;
determining a leakage level based on the leakage data and a predefined ring threshold, wherein the leakage level indicates information about the leakage detected; and
outputting a haptic feedback leak level alert based on the leakage level, wherein the haptic feedback leak level alert discreetly notifies a user about the leakage detected
